(19) United States
(12) Patent Application Publication
(10) Pub. No.: US 2024/0165284 A1
(43) Pub. Date: May 23, 2024

(54) LAMP TO BE INTRODUCED INTO AN OBJECT, IN PARTICULAR FOR ITS DECONTAMINATION

(71) Applicant: CLARANOR, Avignon (FR)

(72) Inventors: Pierre LALUBIN, Graveson (FR); Julien DUBOIS, Rochefort du Gard (FR); Christophe RIEDEL, Avignon (FR)

(21) Appl. No.: 18/262,547

(22) PCT Filed: Jan. 24, 2022
(86) PCT No.: PCT/EP2022/051474

§ 371 Date: Jul. 21, 2023

(30) Foreign Application Priority Data
Jan. 25, 2021 (FR) 2100668

(51) Int. Cl. A61L 2/10

(52) U.S. Cl. CPC A61L 2/10; A61L 2202/11

(57) ABSTRACT

A lamp to be introduced into an object is disclosed, in particular, for decontamination of the object, having a tube with first and second ends, two electrodes fixed in the tube at each of the ends thereof, and an electrical device for supplying the electrodes. The two electrodes have different lengths, one of short length and the other of great length, the electrode of great length extending over a tube length and having an electrode end bearing against the inner wall of the tube at a distance from the second tube end. The lamp also has at least one radial support element fitted around the tube length and having at least two peripheral contacts, on the tube, which are spaced apart from each other by a distance at least twice the diameter of the tube.

The invention relates to a lamp designed to be introduced into a vessel, in particular in order to apply a photonic treatment thereto with the aim of ensuring decontamination of the inside of the vessel by emission of a light flux.

Different techniques are known for decontaminating the inside of vessels, such as preforms, bottles or other flasks, before they are filled with a dietary, cosmetic or pharmaceutical fluid in industrial production and packaging lines.

Patent application FR2774912, for example, describes a method making it possible to sterilize hollow vessels (for example a preform or a bottle): a device for injecting a sterilizing agent, for example hydrogen peroxide, is introduced into the hollow body. After a period of exposure to the sterilizing agent, it is removed by draining the hollow body (when the agent is liquid).

If disinfection using hydrogen peroxide is carried out in the preform before blow moulding, the blow moulding of the preform and the filling are carried out downstream in a sterile environment.

A device and a method are also known from the document GB495499 which ensure the decontamination of the inside of vessels by introducing therein a lamp emitting UV radiation.

The decontamination operation can also be carried out using hot peracetic acid directly in the bottles, after blow moulding. Document FR2917624, for example, describes such an operation. However, it is to be noted that the bottles which undergo such a treatment must be thick enough to withstand the heat. Such a treatment is therefore not suitable for many bottles the weight of which tends to be increasingly reduced and the walls of which are becoming thinner.

Other alternatives are also known for decontaminating/disinfecting hollow bodies: for example, preforms can be disinfected by e-beam processing before blow moulding of the preforms and filling of the bottles. The e-beam is an electron beam which is sprayed onto a surface to be decontaminated. This technology makes it possible to avoid the use of chemical products. However, it should be noted that once the preforms have been decontaminated, the blow moulding and filling of the bottles must always take place in a sterile environment. Document FR3037247 describes an implementation example of such a technology.

Moreover, other embodiments of decontaminating lamps are known, such as those described in US patent U.S. Pat. No. 5,753,996 which proposes a solution for facilitating gripping of the lamp in order to promote its release when it is connected, in order to change it for example for another lamp when it is faulty. Document U.S. Pat. No. 6,013,918 proposes a decontaminating lamp embodiment variant, in particular in the shape of a U, which makes it possible to deactivate microorganisms in a food, pharmaceutical etc. product in order to extend its life.

A purpose of the invention is to propose an alternative to the known devices or methods, which can be implemented equally well for an aseptic application as for ultra-clean application of the vessels/containers.

Moreover, a purpose of the invention is to propose a technical solution which makes it possible to decontaminate the inside of a vessel. By “vessel” is meant any hollow object having an opening for access to the inside of the vessel. It may be, for example, a bottle, a flask, a preform, a jar, a metal can, etc.

Finally, a purpose of the invention is to propose an alternative to the use of chemical agents.

To this end, the invention relates to a lamp to be introduced into an object, as defined by the claims. The lamp is designed in particular for decontaminating said object, comprising:
a tube which extends in a rectilinear direction, having first and second tube ends, capable of diffusing a light flux,
two electrodes fixed inside the tube, each of the two said electrodes being fixed to one end from among the two said first and second tube ends,
an electrical device for supplying the electrodes, electric voltage delivery means between the two said electrodes.

The lamp according to the invention is notable in that:
the two electrodes have different lengths, one of the two electrodes having a short length and being fixed to said first tube end and the other electrode having a long length and being fixed to said second tube end,
said long-length electrode extending over a tube length and having an electrode end located at a distance from said second tube end,
at least one radial support element being fitted around said tube length in which said long-length electrode extends, said at least one radial support element having at least two peripheral points of contact on said tube, said at least two peripheral points of contact being spaced apart from one another by a distance of at least twice the diameter of said tube.

By “electrode” is meant an element or a set of elements capable of generating a plasma and which extends between an electric pulser and an emitter end: in other words, an electrode within the meaning of the invention comprises an element or a set of elements located between a first end (of the element or the set of elements) forming an electric pulser and a second end (of the element or of the set of elements) which is immersed in a gas.

Produced in this way, the invention can be implemented equally well in an industrial production line as in a shop where, for example, it would be proposed to fill containers which have already been used and washed in order to ensure internal decontamination of the vessels before filling them again: the presence of the radial support element makes it possible in particular to fix the lamp in an industrial facility.

The lamp according to the invention can also comprise the following characteristics, separately or in combination:
said at least one radial support element preferably includes at least two peripheral seals and a radial support outer sleeve, said two peripheral seals being positioned between said tube and the sleeve, said sleeve also making it possible to grip said lamp, said at least two peripheral seals corresponding to said at least two peripheral points of contact and being capable of being compressed between said tube and said sleeve,
said sleeve is fitted around said tube and extends:
from the end of said long-length electrode;
at least over said tube length, enclosing said second tube end including said long-length electrode, and
beyond said second tube end in order to form a chamber adjacent to and coaxial with said tube, said chamber at least partially accommodating said electrical device.
the lamp according to the invention advantageously includes an insulating sheath positioned at least partially between said sleeve and said tube, said insulating sheath extending over at least a portion of said tube length including said long-length electrode and extending around said chamber comprising said electrical device,
said electrical device advantageously includes a first rigid and conductive element, to which is fixed said long-length electrode, a second rigid and conductive element, projecting from a free end of the sleeve, said first and second rigid and conductive elements being coaxial, received in said chamber and connected to one another by a flexible electrical cable allowing a radial movement of said first rigid element in said chamber,
the sleeve advantageously includes a portion for manual connection of said lamp to an external device, the external device including a housing for accommodating said portion for manual connection of the sleeve, said portion for manual connection of the sleeve comprising a radial shoulder projecting towards the outside of the sleeve, capable of being positioned against a contact surface of said external device in proximity to said accommodation housing, said shoulder receiving a peripheral seal capable of being compressed against said contact surface of said external device, said connection portion of the sleeve also comprising at least one projecting pin capable of being displaced within an internal guide track/thread arranged in said accommodation housing,
said shoulder preferably has a visual element, capable of coming to face a second visual element borne by said external device when said connection portion of said sleeve is correctly inserted in the accommodation housing of said external device,
said short-length electrode is advantageously connected to said radial support element by means of at least one electrical conductor which runs on the outside of said tube, said radial support element being grounded,
according to an embodiment variant, said short-length electrode is connected to said grounded radial support element by means of at least one mesh of electrical wires which runs on the outside of said tube against its side wall,
preferably, said at least one electrical wire or said mesh is held in position at points on the outer wall of said tube,
the tube is preferably made from quartz.
the light emitted by the lamp is advantageously a decontaminating pulsed light.

The invention also relates to a facility including at least one lamp as defined above, said lamp being mounted in an external device, said external device being mounted mobile in displacement in a direction parallel to the axis of said lamp.

The invention also relates to a facility including at least one lamp as defined above, said lamp being mounted in an external device, said facility including at least one support for a hollow object, said support being mounted mobile in displacement in a direction parallel to the axis of said lamp.

Other advantages and characteristics of the invention will become apparent on examining the detailed description of an embodiment which is in no way limitative, and from the attached drawings, in which:

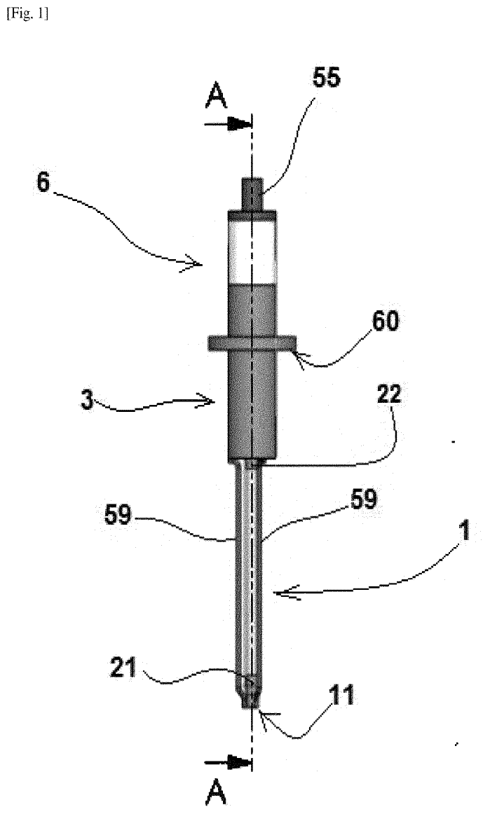

FIG. 1 is a side view of a lamp according to the invention,

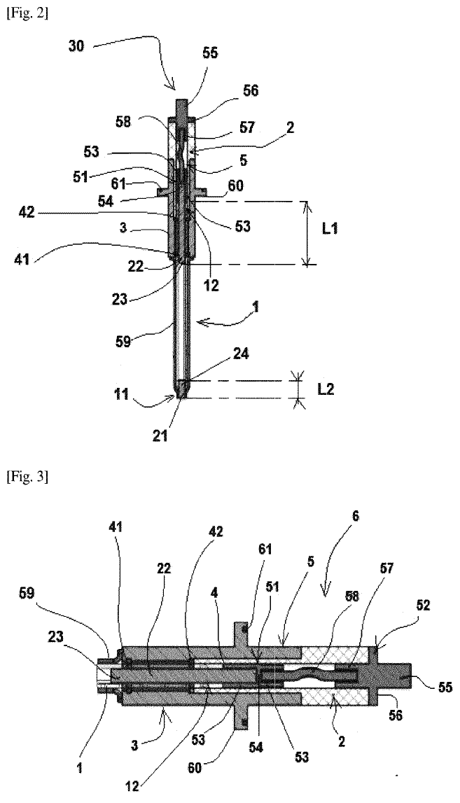

FIG. 2 is an axial cross-section view A-A of the lamp shown in [FIG. 1],

FIG. 3 is an enlargement of a portion of the lamp shown in [FIG. 2],

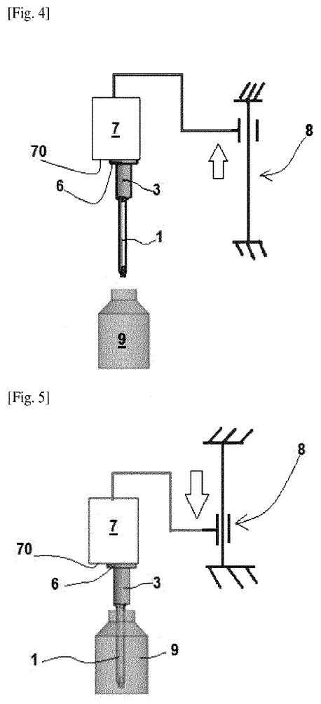

FIG. 4 is a side view of a facility according to the invention, the lamp being mounted on a mobile device, the lamp being illustrated outside an object,

FIG. 5 is a side view of the facility shown in [FIG. 4]; the lamp being inserted in the object, and

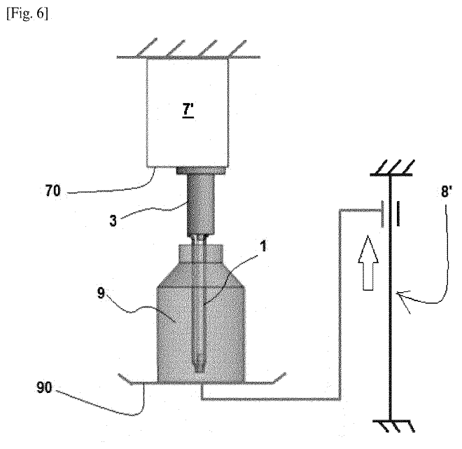

FIG. 6 is a side view of another facility according to the invention, where the device bearing the lamp is fixed and where the object is positioned on a mobile support.

FIGS. 1 to 3 illustrate a lamp according to an embodiment according to the invention.

According to this embodiment, the lamp is designed to decontaminate the inside of objects such as bottles, or other hollow vessels having an opening.

It should be understood that the invention is not limited to a decontamination lamp and that it extends to the manufacture of any lamp which would correspond to the definition of the invention.

The lamp shown in [FIG. 1] as a whole comprises a straight tube 1, which extends in a rectilinear direction, capable of diffusing a light flux.

The shape of the tube that the lamp includes is essential so that the light radiation that it emits can reach the bottom of the vessel in which it is inserted. For example, in the context of a decontamination of a flask of the bottle type, taller than it is wide, the tube of the lamp must be sufficiently long so that, when it is deep in in the flask, the tube end is in proximity to the bottom of the flask.

The tube 1 preferably has a circular cross section, but a tube could be envisaged having a square or rectangular cross section without exceeding the scope of the invention.

A tube 1 having a circular cross section will nevertheless be easier to insert into the circular-section neck of a bottle.

The tube 1, also shown in [FIG. 2], includes a first end 11 and a second end 12.

The first tube end 11 is free (i.e. it is not connected to anything). It is closed by the same material from which the side wall of the tube is made.

In the context of this embodiment, the tube is made from quartz, and the light emitted by the tube is a decontaminating pulsed light, such as a UV-rich, high-power radiation.

The lamp according to the invention, according to other embodiments and other modes of application, could be made from glass (another material allowing a light radiation to pass through) and the radiation emitted by the lamp could be different from a UV radiation, without, however, exceeding the scope of the invention.

The end 11 of the tube 1 is thus produced closed by a wall allowing the decontaminating radiation to pass through. Thus, when this end 11 of the tube 1 is placed in an object (i.e. inside an object), the radiation also passes through this end, as close as possible to the bottom of the object.

The end 12 of the tube is not free, unlike the free end 11.

In order to allow the emission of a radiation, the tube 1 includes two electrodes, fixed inside the tube 1: a first electrode 21 is fixed in proximity to the first tube end 11 and a second electrode 22 is fixed in proximity to the second tube end.

The lamp also includes a device 2 for supplying the electrodes with electricity, which carries the electric current (current delivery means) to the electrodes ([FIG. 2]).

As can be seen in particular in [FIG. 2], the two electrodes do not have the same length: the electrode 21, located at the free end 11 of the tube 1 is shorter than the electrode 22 located close to the end 12 of the tube 1.

The electrode 22 extends over a length L1 of tube 1 which is longer: in the context of the example illustrated, the electrode 22 is up to seven times longer (or substantially up to seven times longer) than the electrode 21 which, itself, extends over a length L2 of tube (in [FIG. 2], the length L1 is three times longer—or substantially three times longer—than the length L2).

It is noted that the free end of the electrode 22 is flush with the end of a sleeve 3 (presented below) and the electrode extends between the end of the sleeve 3 and the end 12 of the tube 1.

The length L1 corresponds to a long tube length while the length L2 corresponds to a short tube length.

The second electrode 22, which extends over the long length L1 of tube 1, has an electrode head 23—the first electrode 21 also comprises an electrode head 24.

The second electrode 22 extends between an electrode head 23 at its free end, the electrode head 23 being located at a distance from the end 12 of the tube 1 and from its other electrode end which acts as a pulser: this distance corresponds to the long length L1 of tube over which the second electrode 22 extends.

It will be understood that the electrode 23 includes all the elements comprised between the electrode head 23 (at a first end) and the portion forming a pulser (at its other end, placed at the tube end).

According to the invention, the lamp also comprises a radial support element outside the tube 1, fitted around the tube 1 over the tube length L1 accommodating the second electrode 22.

In the context of the embodiment shown in FIGS. 1 to 3, this radial support element includes an external radial support sleeve 3, which extends at least over the tube length L1: it thus encloses the portion of the tube 1 in which the second electrode 22 extends, as well as the end 12 of the tube 1.

The sleeve 3 thus offers protection at the end 12 of the tube and to the portion of the tube 1 including the electrode 22. It provides external stiffening to the tube, thus preventing it (for example) from deforming over the length L1: the tube can thus be mounted, for example on a carousel in the context of an industrial production line, because the sleeve 3 partially protects the tube and provides an additional stiffness over a tube length: the tube is thus held over a segment.

Holding the tube over an entire segment makes it possible to implement the lamp in an industrial facility, of the carousel type, because holding over a segment avoids a possible loss of coaxiality: in fact, when an element is held at a single point on a carousel driven in rotation, it can be the case that centrifugal force has an effect on the coaxiality of this element (its end can, for example, come out of alignment). By holding the object (in this case the tube) not at a point, but over a segment, the loss of coaxiality is avoided. The sleeve thus ensures this function.

The sleeve 3 is also useful for fixing the lamp in machines, such machines making it possible for example to move the lamp in an axial direction, in order to introduce it into a hollow vessel or remove it from the vessel.

Finally, by producing a sleeve and a tube the diameters of which are smaller than that of a vessel neck, it is possible to introduce into the vessel not only the portion of the tube which emits a decontaminating flash, but also the sleeve, wholly or partially, which makes it possible to insert the lamp as deeply as possible into the vessel, so that the tube end reaches an area close to the bottom of the vessel, which ensures good decontamination (at least better than if the tube end is located at a distance from the bottom).

In the context of this embodiment, the sleeve 3 does not directly touch the wall of the tube: two peripheral O-ring seals 41 and 42 are also fitted around the tube 1, at least over the tube length L1, between the wall of the tube 1 and the wall of the sleeve 3.

The two O-ring seals 41 and 42 are spaced apart from one another by a distance of at least twice the diameter of the tube 1.

The two O-ring seals 41 and 42 are elastically deformable and have several functions: they act as bearing points on the tube, allowing a slight movement of the tube 1 (over the length L1—by compression) in the sleeve 3 and they allow sealing between the potentially damp outside of the lamp and the inside of the lamp. They thus make it possible to maintain a dry environment in the lamp support to avoid the leakage paths becoming shortened.

An insulating sheath 4 can also be provided between the sleeve 3 and the tube 1, as can be seen in particular in [FIG. 3].

As can be seen in particular in [FIG. 2], the sleeve 3 extends beyond the end 12 of the tube 1 and thus makes it possible to form a chamber 5 that is axially adjacent to the portion of the sleeve 3 which accommodates the end 12 of tube 1.

The chamber 5 accommodates at least a portion of the electrical supply device 2 of the electrodes of the lamp (see FIGS. 2 and 3).

The insulating sheath 4 is positioned at least partially between the tube 1 and the sleeve 3. It extends around the chamber 5 and over at least a portion of the tube 1 which accommodates the electrode 22.

Reference will now be made to the electrical supply device 2 of the electrodes of the lamp.

As can be seen in FIGS. 2 and 3, said electrical supply device 2 includes a first rigid and conductive element 51, to which is fixed said long-length electrode 22, as well as a second rigid and conductive element 52, projecting from a free end of the sleeve 3 ([FIG. 3]).

The rigid and conductive elements 51 and 52 are received in the chamber 5, placed at the ends of the chamber 5, and they are conductive and coaxial. The first rigid element 51 is formed in a single piece by two coaxial hollow cylinders 53 connected by a solid radial wall 54. The second rigid element 52 is formed by a solid cylinder 55, having a radial shoulder 56 and firmly fixed to a hollow cylinder 57 at one of its ends.

The two rigid and conductive elements 51 and 52 are received in the chamber 5 and they are connected to one another by a flexible electrical cable 58, allowing a radial movement of said first rigid element 51 in said chamber 5.

This movement makes it possible to correct an error of coaxiality between the second electrode 22 and the axis of the tube 1. In fact, it is often the case that the electrode 22 is slightly offset because its fastening is carried out manually in the tube.

The electrical supply of the second electrode, fixed in the first rigid element 51, is thus carried out via the outside of the lamp, by means of the second rigid element which comes out of the free end 30 of the sleeve 3.

In order to make it possible to generate a voltage difference between the electrodes, provision is made to connect the first electrode 21 to the sleeve 3 (thus to the radial support element) by means of at least one electrical wire 59 (particularly visible in FIGS. 1 to 3) which runs on the outside of the tube 1 against its side wall.

It should be noted that the sleeve 3 is grounded.

In an advantageous embodiment which is not shown but which would also correspond to the invention, it is possible to provide three electrical wires 59, running on the outside of the tube 1 from the short-length electrode 21 up to said grounded radial support element (the sleeve 3 in the example herein).

According to yet another embodiment, a rigid, conductive strip spaced apart from the tube could be provided, which would ensure the connection between the radial support and the electrode 21. This conductive strip could extend in a direction parallel to the axis of the tube, or in a direction different to a parallel direction for example in order to adapt to technical constraints inherent in a given application.

According to yet another embodiment variant, instead of one or three wires outside the tube 1, provision could be made for the short-length electrode 21 to be connected to the sleeve 3 (thus to the grounded radial support element) by means of at least one mesh of electrical wires which would run outside the tube 1 against its side wall. Such an embodiment would make it possible to contain the pieces of a broken or chipped tube, which would prevent pieces of a broken or chipped tube from falling into a hollow vessel in which the lamp according to the invention would be introduced.

Advantageously, in order to ensure the electrical wire is held in place against the tube 1 (or electrical wires—mesh or not) during the introduction and the removal of the lamp, provision is made for the electrical wire or wires or the mesh to be held at points on the outside wall of said tube (i.e. held by holding points), for example by means of rings slipped around the tube. At the level of the electrode (anode), a holding ring is provided that is larger than the others in order to facilitate starting (in the case of this flash lamp).

Reference will now be made to characteristics of the invention which make it possible to supply the lamp with energy (to switch it on, to connect it to the power grid).

FIG. 4 for example shows an external device 7, to which the lamp shown in FIGS. 1 to 3 is connected.

The external device 7 can be a device fixed on a carousel or on a machine 8 which ensures a movement of the lamp, in order to introduce it into or remove it from a vessel 9 (any hollow object having an opening) and to accompany the movement of the vessel on an industrial production line, for example.

The sleeve 3 includes a portion 6 for manual connection of said lamp to the external device (see FIGS. 1 and 3): in fact, one of the advantages of the invention is that the lamp can be assembled and disassembled on the external device 7 without tools, which is a saving of time when the lamp has to be replaced or disassembled for inspection.

The external device 7 includes a housing for accommodating said manual connection portion 6 of the sleeve 3.

As can be seen in particular in [FIG. 1], the connection portion 6 comprising a shoulder 60 that is radial and projecting towards the outside of the sleeve 3, which is capable of being positioned against a contact surface 70 of said external device 7 (see FIG. 4 or 5).

The shoulder radially receives a peripheral O-ring seal 61, which is capable of being compressed against said contact surface 70 of said external device 7.

This seal serves to seal the electrical compartment.

The connection portion 6 of the sleeve 3 can comprise projecting pins, capable of moving in a guide track arranged in said accommodation housing of the external device 7. The guide track is preferably helical.

Such an embodiment with pins makes it possible to tighten and loosen the fastening of the lamp on the external device, which leads to the compression of the peripheral seal 61 on the surface 70 of the device 7, with a view to ensuring the sealing of the lamp.

Assembly thus does not require any tools and limits the coaxial forces on the lamp which could lead to impacts, breaking of quartz or be the cause of injuries.

In order to allow someone to visualise a good connection of the lamp to the external device, provision is made for the shoulder 60 to have a visual element, capable of coming to face a second visual element borne by said external device when said connection portion of said sleeve is correctly inserted in the accommodation housing of said external device.

Such a visual element can be a pattern that is raised (or not), representing for example an arrow.

It is thus sufficient, to connect the portion 6 to the external device, to insert the portion 6 in the housing by inserting the pins in the tracks that are internal to the housing, then to push the portion 6 into the housing by turning until the markers (visual elements borne by the external device) come to face visual markers of the connection portion 6.

It is thus understood how, without having to use any tool, it is easy to connect a lamp according to the invention to an external device capable of supplying the lamp with energy.

As was explained previously, the external device makes it possible not only to supply the lamp with electrical energy (to diffuse the light radiation) but also to drive the displacement of the lamp, for example in order to insert it into a hollow object (such as a bottle) or to remove it from the object, or to guide the object in a displacement movement.

A facility is thus intended in which the lamp shown in FIGS. 1 to 3 is mounted in an external device 7 shown in FIGS. 4 and 5, in which the external device 7 is mounted mobile in displacement in a direction parallel to the axis of said lamp (for a raising and lowering movement of the lamp).

Another facility is also intended, shown in [FIG. 6], in which the lamp is mounted in an external device 7′, the facility including at least one support 90 for a hollow object 9, said support 90 being mounted mobile in displacement in a direction parallel to the axis of said lamp.

The movement in displacement of the support 90 is ensured by a machine 8′.

In such a facility, the device 7′ is immobile: it is the object which is displaced in order to ensure the introduction of the lamp therein.

It is understood from the preceding description how the lamp according to the invention is easy to manipulate and to connect to an external device.

It should be understood that the lamp could be equipped with an energy supply system comprising a battery, or an electrical wire for connection to the grid, without exceeding the scope of the invention, for a manual use of the lamp.

Thus, the lamp according to the invention can be used either manually or in an automated fashion by being fixed on a machine. In the latter case, it is understood that the lamp can easily be manually connected without tools, which allows a saving of time when it is necessary to change the lamp.

1. A lamp to be introduced into an object, in particular for decontaminating said object, comprising:
a tube which extends in a rectilinear direction, having first and second tube ends, capable of diffusing a light flux;
two electrodes fixed inside the tube, each of the two said electrodes being fixed to one end from among the two said first and second tube ends;
an electrical device for supplying electrodes, voltage delivery means between the two said electrodes;
the two electrodes have different lengths, one of the two electrodes having a short length and being fixed to said first tube end and the other of the electrodes having a long length and being fixed to said second tube end;
said long-length electrode extending over a tube length and having an electrode end located at a distance from said second tube end;
at least one radial support element being fitted around said tube length in which said long-length electrode, said at least one radial support element having at least two peripheral points of contact on said tube, said at least two peripheral points of contact being spaced apart from one another by a distance equal to at least twice the diameter of said tube;
in that said at least one radial support element includes at least two peripheral seals and a radial support outer sleeve, said two peripheral seals being positioned between said tube and the sleeve, said sleeve also making it possible to grip said lamp, said at least two peripheral seals corresponding to said at least two peripheral points of contact and being capable of being compressed between said tube and said sleeve, and in that said sleeve is fitted around said tube and extends:
from the end of said long-length electrode;
at least over said length of tube, enclosing said second tube end including said long-length electrode.

2. The lamp according to claim 1, characterized in that said sleeve extends:
beyond said second tube end in order to form a chamber adjacent to and coaxial with said tube, said chamber at least partially accommodating said electrical device.

3. The lamp according to claim 1, characterized in that it includes an insulating sheath positioned at least partially between said sleeve and said tube, said insulating sheath extending over a least a portion of said tube length including said long-length electrode and extending around said chamber comprising said electrical device.

4. The lamp according to claim 2, characterized in that said electrical device includes a first rigid and conductive element, to which is fixed said long-length electrode, a second rigid and conductive element, projecting from a free end of the sleeve, said first and second rigid and conductive elements being coaxial, received in said chamber and connected to one another by a flexible electrical cable allowing a radial movement of said first rigid element in said chamber.

5. The lamp according to claim 1, characterized in that the sleeve includes a portion for manual connection of said lamp to an external device, the external device including a housing for accommodating said portion for manual connection of the sleeve, said portion for manual connection of the sleeve comprising a radial shoulder WO projecting towards the outside of the sleeve, capable of being positioned against a contact surface of said external device in proximity to said accommodation housing, said shoulder receiving a peripheral seal capable of being compressed against said contact surface of said external device, said connection portion of the sleeve also comprising at least one projecting pin capable of being displaced within an internal guide track/thread arranged in said accommodation housing.

6. The lamp according to claim 5, characterized in that said shoulder has a visual element, capable of coming to face a second visual element borne by said external device when said connection portion of said sleeve is correctly inserted in the accommodation housing of said external device.

7. The lamp according to claim 1, characterized in that said short-length electrode is connected to said radial support element by means of at least one electrical conductor which runs on the outside of said tube, said radial support element being grounded.

8. The lamp according to claim 1, characterized in that said short-length electrode is connected to said grounded radial support element by means of at least one mesh of electrical wires which runs on the outside of said tube against its side wall.

9. The lamp according to claim 7, characterized in that said at least one electrical wire or said mesh is held in position at points on the outer wall of said tube.

10. The lamp according to claim 1, characterized in that the tube is made from quartz.

11. The lamp according to claim 1, characterized in that the light emitted by the lamp is a decontaminating pulsed light.

12. A facility including at least one lamp according to claim 1, said lamp being mounted in an external device, said device being mounted mobile in displacement in a direction parallel to the axis of said lamp.

13. A facility including at least one lamp according to claim 1, said lamp being mounted in an external device said facility including at least one support for a hollow object, said support being mounted mobile in displacement in a direction parallel to the axis of said lamp.
